(19) United States
(12) Patent Application Publication
(10) Pub. No.: US 2024/0159693 A1
(43) Pub. Date: May 16, 2024

(54) Target for calibrating and determining the spatial resolution, SNR and/or CNR associated with an XCT system

(71) Applicant: The University of Manchester, Manchester, Greater Manchester (GB)

(72) Inventors: Tristan James Lowe, Manchester, Greater Manchester (GB); Andrew Francis Bowfield, Manchester, Greater Manchester (GB); Samuel Johnston, Manchester, Greater Manchester (GB)

(21) Appl. No.: 18/549,848

(22) PCT Filed: Mar. 9, 2022
(86) PCT No.: PCT/EP2022/056050

§ 371 Date: Sep. 8, 2023

(30) Foreign Application Priority Data
Mar. 10, 2021 (GB) 2103323.8

(51) Int. Cl. G01N 23/046; A61B 6/03; A61B 6/58

(52) U.S. Cl. CPC G01N 23/046; A61B 6/032; A61B 6/582; G01N 2223/3037

(57) ABSTRACT

A target is provided for calibrating and determining the spatial resolution, SNR and/or CNR associated with an X-ray computed tomography system having a rotation axis about which a beam of X-rays is rotated relative to the target to produce signals that are processable to generate an image on an imaging plane perpendicular to the rotation axis. The target comprises a radiopaque body and is configured to be locatable in the system with a centre of the body on the rotation axis. A thickness direction of the body parallel to the rotation axis such that the body extends radially from its centre in the imaging plane. The body contains a plurality of non-radiopaque columns extending longitudinally in the thickness direction of the body. The columns are arranged in first sub-groups of identically-shaped columns with the columns of each first sub-group sharing a respective predetermined transverse diameter. The first sub-groups are spaced from each other, and the columns of each first sub-group are also spaced from each other. The first sub-groups are members of one or more first sets. The first sub-groups of each first set are arranged such that within that first set the first sub-groups are at respective and different radial distances from the centre, and within that first set the predetermined transverse diameters of the first sub-groups vary with distance from the centre. An image of the target generated by the system can be used to calibrate and determine the spatial resolution, SNR and/or CNR associated with the system on the basis of the predetermined column transverse diameters.

### FIELD OF THE INVENTION

The present invention relates to a target for calibrating and determining the spatial resolution, signal/noise ratio (SNR) and/or contrast noise ratio (CNR) associated with an X-ray computed tomography (XCT) system.

### BACKGROUND

Since the 1990's XCT has become an increasingly popular method for non-destructive evaluation (NDE) in both industry and academia. XCT is in essence a simple imaging technique that utilises X-ray attenuation of the test specimen. During an XCT scan the test specimen is positioned between an X-ray source and detector. In conventional lens-less cone beam XCT the geometry (cone) of the X-ray optics means that the image is magnified by a scale factor given by the geometric magnification of the system. The geometric magnification M is related to Focal Spot to Object Distance (FOD) and X-ray Focal spot to Detector Distance (FDD) by the expression M=FDD/FOD. The effective pixel size P_{eff} of the magnified image is given by P_{eff}=Pₚ/M where Pₚ represents the pixel pitch of the detector array.

In XCT the reconstructed voxel size is equivalent to the effective pixel size of the constituent radiographs. The correct effective pixel size is reliant on both the FDD and FOD of the system being correctly calibrated.

In practice, the FOD is a known distance for a given manipulator position for the specimen stage, but direct measurement of the FDD is difficult due to both the X-ray source being located behind a window on the X-ray gun and the detector scintillator being located behind a number of protective layers. However magnification can be determined using spheres of known dimensions in order to determine a system's correct FDD value. For example, a sphere fitting algorithm can be applied to reconstructed images of calibrated ball bar targets to quantify the centre to centre distance of the spheres on the target. The calibration target is scanned at a set of distances between the X-ray source and detector to produce a calibration curve for all source-sample distances. These measurements are then used to calculate the correct source to detector distances using the expression L=LᵥₒₓP_{eff}FOD/FDD, where L is the calibrated sphere centre to sphere centre distance, size and Lᵥₒₓ is the measured length between the sphere centres in the reconstructed volume in voxels. The corrected source to detector distance then ensures that the effective pixel size is correctly calculated as a function of magnification, assuming the manipulator position for the FOD is accurately known.

However, such an approach does not take into account nonlinear environmental factors (e.g. temperature and humidity), the influence of the X-ray spectrum, or imperfections in the specimen stage. These can be important for scans requiring high dimensional accuracy. As such, metrology grade XCT instruments conventionally rely on multiple high resolution encoders for accurate FOD measurements and thermally stable materials such as granite for mounting components.

Optimal imaging position of the specimen relative to the X-ray source and detector is also a key factor in XCT. The voxel calibration approach described above relates to the measurement accuracy of features in the post-processing stage, it does not give any information regarding which features can be observed. The spatial resolution, SNR and CNR of the reconstructed data is determined by a combination of factors including the effective pixel size, image noise and contrast. Other factors that can play a part are un-sharpness of the image due to focal spot size and magnification, image distortion and reconstruction.

The data obtained in an XCT acquisition constitute a series of 2D images of a 3D object obtained for different relative angular positions of the X-ray beam and the target. To achieve this either the X-ray beam is held stationary and the object is rotated about an axis, or the beam is rotated about the axis and the object is held stationary. A reconstruction process is then required to generate a 3D representation of the specimen. The reconstruction process itself, due to the use of filters, results in image quality loss from the 2D radiographic data to the virtual 3D volume. The degree of information loss and therefore spatial resolution, SNR and CNR depends upon the severity of filters employed. A second source of information loss is summarised by the Nyquist—Shannon sampling theorem: within an XCT scan the greatest density of information is located at the centre of rotation of the scan, and as the radial distance from the centre of rotation increases there is an increase in information loss.

The growth of 3D printing of components, for example for use in the aviation and automotive industries, has led to increasing adoption of low weight, high strength components utilising complex internal geometries. These components have the potential to both improve efficiency in the production phase and ultimate use, resulting in significant cost savings. However, the very complexity of such components, and defects (such as porosity) that may be introduced as part of the manufacturing process can complicate the issue of quality control. This issue is particularly important for safety-critical components. Thus appropriate non-destructive testing (NDT) of such parts is needed, and XCT is an attractive candidate for this. However, difficulties with accurately and repeatably calibrating and determining spatial resolutions, SNR and CNR of XCT images has impeded more widespread adoption of XCT for NDT.

### SUMMARY OF THE INVENTION

Thus it would be desirable to provide a target that can be used to calibrate and determine important image quality metrics such as spatial resolution, SNR and CNR of reconstructed XCT data. Such a target can allow users to improve the optimisation of the X-ray imaging setup and can improve post-processing feature measurement confidence levels and measurement accuracies.

Accordingly, in a first aspect, the present invention provides a target for calibrating and determining the spatial resolution, SNR and/or CNR associated with an X-ray computed tomography system having a rotation axis about which a beam of X-rays is rotated relative to the target to produce signals that are processable to generate an image on an imaging plane perpendicular to the rotation axis, wherein:
the target comprises a radiopaque body and is configured to be locatable in the system with a centre of the body on the rotation axis, and a thickness direction of the body parallel to the rotation axis such that the body extends radially from its centre in the imaging plane;
the body contains a plurality of non-radiopaque columns extending longitudinally in the thickness direction of the body, the columns being arranged in first sub-groups of identically-shaped columns with the columns of each first sub-group sharing a respective predetermined transverse diameter, the first sub-groups being spaced from each other, and the columns of each first sub-group also being spaced from each other; and
the first sub-groups are members of one or more first sets, the first sub-groups of each first set being arranged such that within that first set the first sub-groups are at respective and different radial distances from the centre, and within that first set the predetermined transverse diameters of the first sub-groups vary with distance from the centre;
whereby an image of the target generated by the system can be used to calibrate and determine the spatial resolution, SNR and/or CNR associated with the system on the basis of the predetermined column transverse diameters.

References herein to non-radiopaque columns contained in a radiopaque body are intended to refer to columns formed in the body that have a degree of radio-opacity that is less than that of the body itself. That is, references herein to non-radiopaque columns refer to radiolucent columns. Thus, material of the body should be more opaque to X-rays or other radiation than each of the columns contained in the body. For example, the body should have a higher Hounsfield unit (HU) value (CT number) than each of the columns formed in the body. The columns may, for example, be filled with a gas, such as air.

Thus, in an aspect, the present invention provides a target for calibrating and determining the spatial resolution, SNR and/or CNR associated with an X-ray computed tomography system having a rotation axis about which a beam of X-rays is rotated relative to the target to produce signals that are processable to generate an image on an imaging plane perpendicular to the rotation axis, wherein:
the target comprises a radiopaque body and is configured to be locatable in the system with a centre of the body on the rotation axis, and a thickness direction of the body parallel to the rotation axis such that the body extends radially from its centre in the imaging plane;
the body contains a plurality of radiolucent columns extending longitudinally in the thickness direction of the body, the columns being arranged in first sub-groups of identically-shaped columns with the columns of each first sub-group sharing a respective predetermined transverse diameter, the first sub-groups being spaced from each other, and the columns of each first sub-group also being spaced from each other; and
the first sub-groups are members of one or more first sets, the first sub-groups of each first set being arranged such that within that first set the first sub-groups are at respective and different radial distances from the centre, and within that first set the predetermined transverse diameters of the first sub-groups vary with distance from the centre;
whereby an image of the target generated by the system can be used to calibrate and determine the spatial resolution, SNR and/or CNR associated with the system on the basis of the predetermined column transverse diameters.

Contrast variation on radial line profiles which intersect through the columns can thus be used for calibration and spatial resolution, SNR and/or CNR determination. Meanwhile, the target can have similar X-ray attenuation properties to that of an object to be imaged by the XCT system, and the columns can be sized to emulate the porosity or other feature or defect in the object that it is desired to measure, quantify or image. The calibration and spatial resolution (or SNR/CNR) determination can thus be performed on a target which emulates the object, whereby the XCT system can perform accurate and repeatable NDT on the object.

The columns of each first sub-group may be spaced from their nearest neighbour or neighbours by a distance which is in a range from the respective predetermined transverse diameter to three times the respective predetermined transverse diameter, and which is preferably equal to the respective predetermined transverse diameter. This is consistent with using Michelson contrast along a given line profile to determine resolution limits and for calibration.

Conveniently, the columns may be holes extending in the thickness direction, and preferably are through-holes extending across the entire thickness of the body. The columns may thus be filled with ambient air. The holes are relatively robust, and through-holes in particular, can be easily cleaned, e.g. in a sonic bath.

The columns of each first sub-group may share a respective predetermined length in the thickness direction. A given line profile through columns of a first sub-group should then intersect all the columns in its path. The respective predetermined length may be at least three times the respective predetermined transverse diameter. In this way, a line profile can avoid being too close to the ends of the columns, thereby reducing end effects on the Michelson contrast.

Conveniently, the body may be planar, and preferably is disc-shaped. An axisymmetric target is consistent with reconstructions codes that reconstruct a cylindrical image.

The first sub-groups of each first set may be arranged at respective non-periodic angular positions around the centre of the body. Typically, in this arrangement there is only one first set. The arrangement can reduce the number of columns encountered by an X-ray photon along a given ray path, decreasing the interference in the signal response arising from different first sub-groups.

Alternatively, the first sub-groups of each first set may be arranged along a respective, radially extending, first group line. Typically, in this arrangement there are plural first sets, and thus plural first group lines. The arrangement conveniently allows contrast variation on line profiles taken along the first group lines to be used for calibration and spatial resolution (or SNR/CNR) determination, thereby reducing the number of line profiles needed. For example, the target may have three first group lines which are formed in a tristar-shape such that the three first group lines are angularly spaced 120° apart. Such a target can have a relatively low number of columns and thus can better emulate the X-ray attenuation properties of a solid object to be imaged having little or no voids or porosity. In another example, the target may have four first group lines which are formed in a cross-shape such that the four first group lines are angularly spaced 90° apart. In this way, line profiles can be obtained along four radial directions, facilitating exploration of any astigmatism in the XCT system.

The first sub-groups may be arranged within each first set (i.e. along the one or more first group lines when each first set is arranged along a respective, radially extending, first group line) such that the predetermined transverse diameters of the first sub-groups decrease with increasing distance from the centre of the body. This compliments the Nyquist-Shannon theorem whereby the achievable spatial resolution (and SNR/CNR) deteriorates as a function of distance away from the centre of the target. The first group lines thus allow quantification of the number of XCT projections required to resolve a feature of a predetermined size.

The body may further contain a plurality of further non-radiopaque (radiolucent) columns extending longitudinally in the thickness direction, the further columns being arranged in second sub-groups of identically-shaped columns with the columns of each second sub-group sharing a respective predetermined transverse diameter, the second sub-groups being spaced from each other, and the columns of each second sub-group also being spaced from each other. However, unlike the first sub-groups, these second sub-groups are members of one or more second sets, the second sub-groups of each second set being arranged such that within that second set the second sub-groups are at respective and different radial distances from the centre, and within that second set the predetermined transverse diameters of the second sub-groups increase with increasing distance from the centre. The second sub-groups of each second set may be arranged along a respective, radially extending, second group line. The second sub-groups enable a comparison of the measurement confidence of similarly sized columns at different radial positions to be made. In this way it is possible to determine if a given feature is unresolved because it is too small or if it is too far from the centre of the body and thus susceptible to under-sampling information loss. The further non-radiopaque (radiolucent) columns may be holes extending in the thickness direction, and preferably are through-holes.

The columns of each sub-group (i.e. first sub-group or second sub-group) may be arranged in first and second column rows, the first column row extending along a radial direction of the body, and the second column row extending perpendicularly thereto. Line profiles along column rows can then be taken in both radial and azimuthal directions, allowing calibration and spatial resolution (or SNR/CNR) determination in both radial and azimuthal directions. For example, the first and second column row of each sub-group may form a cross-shape.

Conveniently, each column (whether of a first sub-group or a second sub-group) may be square prismatic, and thereby can intersect as a square cross-section on the imaging plane. The predetermined transverse diameter of each column is then equated to the length of the sides of its square cross-section. The flat sides of square prismatic columns enable easier identification of column to non-column transitions on a line profile. The two opposing sides of the square cross-section are typically perpendicular to a radial direction of the body.

One or more regions of the body may be perforated with a regular pattern of apertures. Conveniently, the apertures may be tessellating polygonal (e.g. hexagonal) apertures. The number, spacing and/or size of the apertures can be adjusted so that X-ray transmissibility through the target is similar to that of a porous or void-containing object to be imaged by the X-ray computed tomography system.

In a second aspect, the present invention provides a combination of an X-ray computed tomography system having a rotation axis about which a beam of X-rays is rotated relative to the target to produce signals that are processable to generate an image on an imaging plane perpendicular to the rotation axis, and the target according to the first aspect for calibrating and determining the spatial resolution, SNR and/or CNR associated with the system.

In a third aspect, the present invention provides use of the target according to the first aspect for calibrating and determining the spatial resolution, SNR and/or CNR associated with an X-ray computed tomography system having a rotation axis about which a beam of X-rays is rotated relative to the target to produce signals that are processable to generate an image on an imaging plane perpendicular to the rotation axis.

In a fourth aspect, the present invention provides a method for imaging an object using an X-ray computed tomography system having a rotation axis about which a beam of X-rays is rotated relative to the target to produce signals that are processable to generate an image on an imaging plane perpendicular to the rotation axis, the method including performing the steps of:
(a) providing the target according to the first aspect, and an object to be imaged, the target being formed of a material which has substantially the same attenuation properties to passage of X-rays therethrough as the material of the object;
(b) locating the target in the system with the centre of its body on the rotation axis, and the thickness direction of the body parallel to the rotation axis such that the body extends radially from its centre in the imaging plane, and then generating an image of the target using the system to calibrate and determine the spatial resolution, SNR and/or CNR associated with the system; and
(c) locating the object in the system, and then generating an image of the object using the system;
wherein steps (b) and (c) can be performed in any order, and the calibration and spatial resolution, SNR and/or CNR determination resulting from step (b) is applied to the image generated at step (c).

When the object is porous or contains voids, in step (a) the body of the target may be perforated with a regular pattern of apertures, such as tessellating polygonal (e.g. hexagonal) apertures, so that X-ray transmissibility through the target is similar to that of the object. For example, the number, spacing and/or size of the apertures can be adjusted to achieve similar X-ray transmissibility.

In step (a) the body of the target may be provided with an imaging structure having a similar or substantially the same shape to that of the object, e.g. so that X-ray transmissibility through the target is similar to that of the object. Correspondingly, in the first aspect, the body may comprise an imaging structure having a similar or substantially the same shape to that of an object to be imaged.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a plan view of an example target for calibrating and determining the spatial resolution, SNR and/or CNR associated with an XCT system;

FIG. 2 shows in detail non-radiopaque (radiolucent) columns of one of sub-group of columns of the target of FIG. 1;

FIG. 3 shows a plan view of a variant of the target of FIG. 1;

FIG. 4 shows a plan view of a further variant of the target of FIG. 1;

FIG. 5 shows a plan view of another variant of the target;

FIG. 6 shows a plan view of another variant of the target;

FIG. 7A shows an XCT image of a section through a complex aerofoil produced by additive manufacture;

FIG. 7B shows a close-up view of the leading edge part of the section of FIG. 7A with porosity identified in red;

FIG. 8 shows a line profile along the radial direction occupied by a line of column sub-groups of the target of FIG. 1;

FIG. 9 shows a plot of spatial resolution in radial and azimuthal directions as a function of number of projections used for XCT reconstruction;

FIG. 10A shows a plan view of another variant of the target; and FIG. 10B shows a plan view of another variant of the target;

FIG. 11 is a photograph of a 3D printed target;

FIG. 12 is a photograph of another 3D printed target;

FIG. 13 is a photograph of another 3D printed target;

FIG. 14 is a photograph of a titanium target;

FIG. 15A shows a plot of MTF versus feature size; and FIG. 15B shows a corrected plot of MTF versus feature size;

FIG. 16 illustrates how data density can vary with radial distance; and FIG. 17 illustrates how data density and X-ray intensity can vary with radial distance;

FIG. 18A shows a comparison of X-ray path lengths for a turbine blade to be imaged and a fully dense target; FIG. 18B shows a comparison of X-ray path lengths for the turbine blade and the target of FIG. 10A; and FIG. 18C shows a comparison of X-ray path lengths for the turbine blade and the target of FIG. 10B;

FIG. 19A illustrates image quality for the target of FIG. 12; and FIG. 19B illustrates image quality for the target of FIG. 13; and

FIG. 20A illustrates a reconstruction of a bead pore; and FIG. 20B illustrates a reconstruction of a column of the target of FIG. 12.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Description of Target

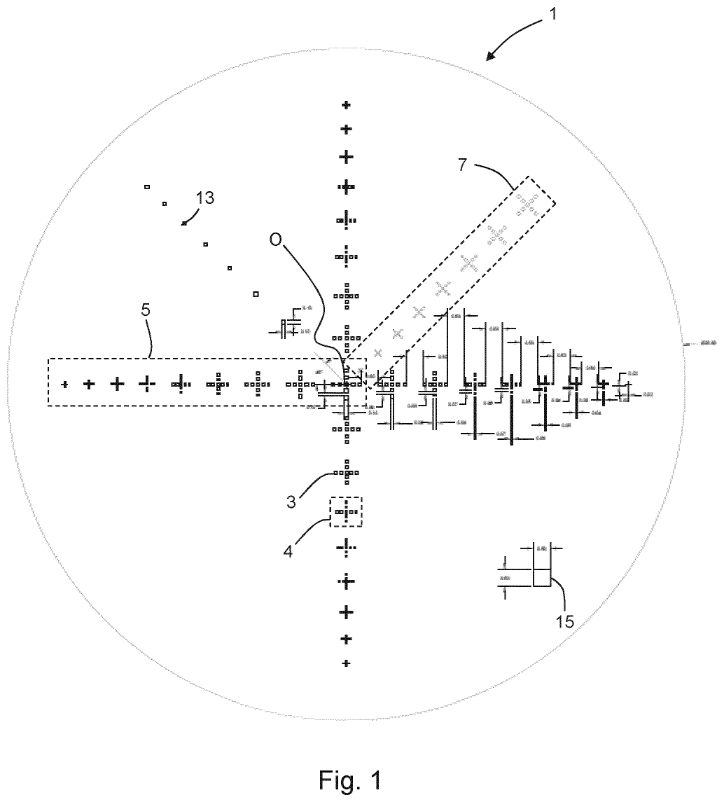

FIG. 1 shows a plan view of an example target for calibrating and determining the spatial resolution (or SNR/CNR) associated with an XCT system. Conveniently, the target comprises a body in the shape of a circular disc 1 formed of a radiopaque material, although other shapes for the body are possible. The fabrication process selected for each target can be determined by the desired target diameter. For a target up to 200 mm in diameter, (a typical size for industrial applications) a larger piece of the respective material can be cut into a disc which is then polished to 1 micron surface roughness. Alternatively, target discs greater than 30 mm in diameter can conveniently be fabricated by additive manufacturing. The good surface finish obtained with this fabrication process can remove the need for the surface to be polished to 1 micron surface roughness.

The final thickness of the disc 1 is generally no less than three times the transverse diameter of the largest non-radiopaque (radiolucent) columns 3 (discussed below) to be formed in the target. These columns can be cut into the polished discs using a laser cutter. Such cutting technology allows columns with transverse diameters of down to about 20 microns to be formed. Columns with transverse diameters of less than 20 microns can be etched using Focused Ion Beam Scanning Electron Microscopy (FIB SEM).

After the columns 3 are formed, a final high resolution polish is performed to clean the surface of the disc 1, and an ultrasonic bath then removes any trapped debris.

The columns 3 can be characterised using SEM or optical microscopy at a resolution which is preferably 1 to 2 orders of magnitude smaller than the smallest columns in the target. For example, with a 20 mm diameter target, the smallest columns may have a diameter of 20 microns, and therefore the pixel size for the SEM or optical analysis may be in the range 2 to 0.2 microns. The actual true size of each column is recorded, and this information can then be used to update a CAD design of the target, providing a unique fingerprint for the target specifying the size and position of each of its columns. This characterisation also ensures that each target conforms to quality and accuracy requirements, and that error analysis for spatial resolution (or SNR/CNR) information and measurement confidence information is unique to each target.

The radiopaque material which forms the disc 1 can have substantially the same attenuation properties to passage of X-rays therethrough as the material of an object which is to be imaged by the system, e.g. the target and the object can be formed of the same material. More particularly, the radiopaque material can be a high grade, familiar engineering material such as titanium, steel, Inconel, aluminium, plastic etc. The Beer-Lambert law relates the attenuation of X-rays to the properties of the material through which the X-rays are travelling, and applies equally to an object to be imaged and the target. Therefore, by forming the target of the same material as the object to be imaged, it is possible to provide a robust process for calibration and spatial resolution (or SNR/CNR) determination that is well-adapted to the needs of that object. For example, if XCT is to be used to characterise pores in a titanium matrix of a component, forming the target of the same titanium matrix and with pores (or equivalent features) of known dimensions can be used obtain highly representative signal responses.

The circular shape of the disc 1 is consistent with reconstructions codes, such as the commonly used FDK (Feldkamp, Davis and Kress) back-projection algorithm, that reconstruct a cylindrical image. However, this does not exclude that the disc can have other shapes, for example non-circular and/or with cut-outs to emulate corresponding features (e.g. cooling channels) in an object to be imaged.

The disc 1 has a centre O, which in use is located on the rotation axis of the XCT system, with the plane of the disc perpendicular to that axis and the thickness direction of the disc parallel to the rotation axis. Thus the disc body extends radially from its centre in the imaging plane of the XCT system. In this example, the disc is 20 mm in diameter.

The disc 1 contains plural non-radiopaque (radiolucent) columns 3 which each extends longitudinally in the thickness direction of the disc. Conveniently, the columns can be holes, e.g. through-holes, formed in the radiopaque material of the disc. When the columns are through-holes, the length of each column is thus just the thickness of the disc. The columns 3 are used for calibration and spatial resolution (or SNR/CNR) determination. Providing imaging contrast features by means of such holes within a radiopaque (typically metallic) matrix is generally a more robust approach than conventional approaches based on embedding highly X-ray attenuating materials, such as gold or tungsten, in a less X-ray attenuating material, such as glass or a polymer. In particular, the holes are not susceptible to distortion if the target is mishandled or dropped, and the holes are easy to manufacture. They can also be cleaned quite easily, e.g. by giving the target a sonic bath.

The columns 3 are arranged in sub-groups 4 (one such sub-group indicated by a respective dashed rectangle in FIG. 1) of identically-shaped columns with the columns of each sub-group sharing a respective predetermined transverse diameter. The sub-groups are spaced from each other, and the columns of each sub-group are also spaced from each other. The sub-groups are members of different sets, the sub-groups of each set being arranged along a respective, radially extending, group line. Within each group line (i.e. within each set), the predetermined transverse diameters of the sub-groups vary with distance from the centre.

For example, the disc 1 has four first group lines 5 (i.e. first sets), one of which is indicated by a respective dashed rectangle in FIG. 1. Each first group line extends from the centre of the disc and together the four first group lines are formed in a cross-shape such that the four first group lines are angularly spaced 90° apart. A central one of the sub-groups 4, centred on the centre O of the disc, is shared by all of the first group lines 5. The (first) sub-groups of each first group line are arranged such that the predetermined transverse diameters of the sub-groups decrease with increasing distance from the centre. The cross configuration results in a higher density of columns 3 towards the centre O of the disc 1, which in turn provides a greater information density for sampling as defined by the Nyquist-Shannon theorem towards the axis of rotation of the XCT system. Comparison of imaging results obtained from the 90° spaced first group lines can provide confidence that the XCT system provides similar imaging characteristics (i.e. calibration, spatial resolution, SNR and CNR) in all angular directions, or that any astigmatism in the system can be identified and quantified.

The disc 1 also has a second group line 7 (i.e. a second set) which extends from the centre of the disc midway between two of the first group lines 5. The (second) sub-groups of the second group line are arranged such that the predetermined transverse diameters of the sub-groups increase with increasing distance from the centre. The second sub-groups of the second group line do not include the sub-group at the centre O of the disc.

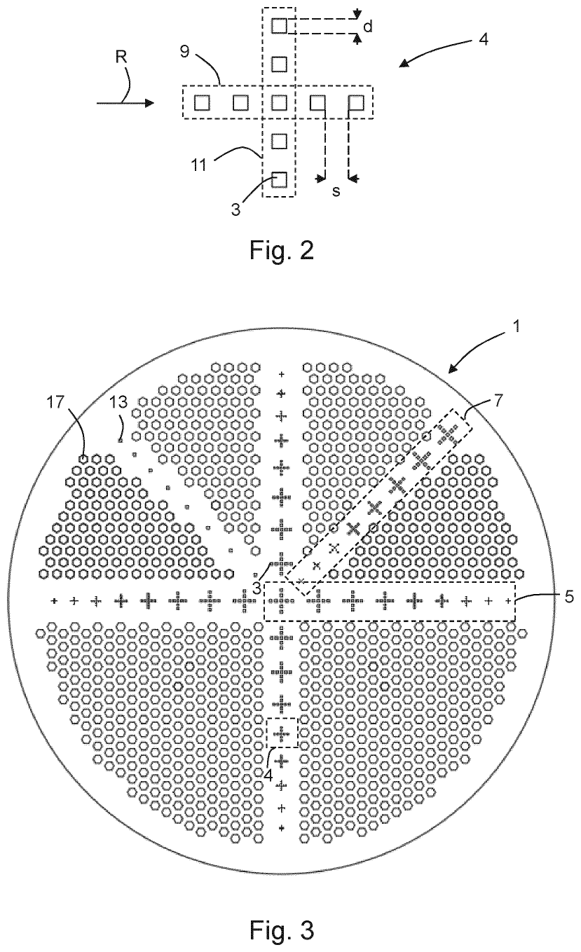

FIG. 2 shows in detail the columns 3 of just one of the (first or second) sub-groups 4. The columns are square prismatic, and thus intersect as a square cross-section on the imaging plane of the XCT system. The flat sides of the square prismatic columns 3 enables easier identification of column to non-column transitions on a line profile through a row of the columns on the image plane. Curved sides would be more difficult to define and identify. In addition, curved sides can exacerbate partial volume effects due to any lack of alignment between the sides and pixel edges.

Each column 3 has transverse diameter d, which is the length of the sides of its square cross-section, and each column is spaced from its nearest neighbour or neighbours by a distance s. The columns are arranged in first 9 and second 11 column rows, indicated by respective dashed rectangles, the first column row extending along a radial direction R of the disc 1, and the second column row extending perpendicularly to that direction. Conveniently, the first and second column rows can thus form a cross-shape, with the central column of the sub-group 4 being shared by both column rows. The columns are further orientated so that two opposing sides of each square cross-section are perpendicular to the radial direction. The second column 11 can be used to identify any imaging changes in the azimuthal direction of the XCT system.

The columns 3 are typically configured so that the length of each column (i.e. its length of extension in the thickness direction on the disc 1) is at least three times its transverse diameter d. Thus when the columns are through-holes in the disc 1, and the disc has a uniform thickness, ensuring that the columns with the biggest transverse diameter d have this length characteristic (which columns are typically the columns of the sub-group 4 at the centre O of the disc), ensures that all the columns share the characteristic.

In the example of FIG. 1, the transverse diameter d of the columns 3 of the sub-group 4 at the centre O of the disc 1 is 0.10 mm, and then along each first group line 5 d decreases progressively in increments of 0.01 mm for each of eight subsequent first sub-groups, the transverse diameter d of the columns of the furthest first sub-group from the centre O being 0.02 mm. The corresponding distances s by which the columns within each first sub-group are spaced from each other are set so that s=d for each first sub-group, i.e. s=0.10 mm at the centre sub-group and 0.02 mm at the furthest sub-group from the centre O. When producing a line profile along a row of columns, having s=d provides a simple, repeatable, periodic pattern between non-radiopaque (radiolucent) and radiopaque locations which facilitates ease of identification and subsequent analysis. However, more generally, s can lie in the range from d to 3d.

For the second group line 7, the transverse diameter d of the columns 3 of the second sub-group 4 closest to the centre O of the disc 1 is 0.02 mm. d then increases progressively in increments of 0.01 mm for each of seven subsequent second sub-groups so that the transverse diameter d of the columns of the second sub-group furthest from the centre O of the disc 1 is 0.09 mm. Again, s=d for each second sub-group, but more generally can lie in the range from d to 3d.

The spacing between sub-groups 4 in each first group line 5 and in the second group line 7 is 0.5 mm.

In addition to the columns 3 which create the sub-groups 4, first group lines 5 and second group line 7, the disc 1 also has an extra line of seven further square cross-section columns 13, the extra line extending radially at 90° from the second group line 7. The further columns 13 all have an identical transverse diameter d of 0.10 mm, with the spacing between the further columns decreasing with increasing distance from the centre so that each further column is located at a radial distance from the centre O which is identical to the radial distance from the centre O of a respective one of the seven outermost first sub-groups of each first group line 5. The further columns are square prismatic through-holes, orientated so that any two opposing sides of each square cross-section are perpendicular to the direction of one of the first group lines 5.

Finally, the disc 1 also has a square through-hole aperture 15 in a quadrant of the disc 1 not occupied by the second group line 7 or the extra line of further columns 13. The square aperture has sides of 0.50 mm length. This relatively large feature can be used to orientate image data obtained from the target in virtual space in order to identify feature positions and facilitate the application of relevant profiles. A shape other than a square can be used, e.g. a triangular aperture can be adopted. However, either way, it is preferable that the shape has flat sides, like the columns 3, 13, which facilitates automated feature identification. The dimensions of the large feature can also be measured by Coordinate Measurement Machine (CMM), SEM or other measuring technique, and the results compared with those derived from the calibration of the XCT system to derive measurement error. By locating the large aperture 15 at a position where it is distal from all other features of the disc, it does not significantly interfere with those other features when they are used for calibration and spatial resolution (or SNR/CNR) determination.

Generally, the target is scalable to the object that is to be scanned by the XCT system. For example, if a turbine blade is 100 mm in size is to be scanned, then a 100 mm diameter disc 1 can be used with appropriately sized columns 3 and further columns 13. On the other hand if the object is 10 mm in size then a 10 mm diameter sample may be used. This scalability takes into account the spatial resolution limitations in XCT due to magnification.

Separately, the target can be modified to better replicate the volume of material in the imaged object along the X-ray photon path. For example, the target of FIG. 1 is fully dense, apart from the columns 3, the further columns 13 and the aperture 15, and thus is best suited for use in association with an imaged object which is also fully dense, apart from the porosity or other defects or features which are the subject of NDT. However, if the object contains relatively macro hollow structures, such as cooling ducts or pipe like structures, which are not themselves the subject of NDT, then a better match with the Beer-Lambert law can be obtained by providing the target with structures to emulate attenuation response of the object. For example, FIG. 3 shows a variant of the target in which the disc 1 is perforated with a regular pattern of hexagonal apertures 17 so that the volume of material along X-ray photon paths is similar to that of a hollow object.

Compared to triangles or squares, the hexagonal apertures 17 reduce streak artefacts, which are an imaging problem associated with sharp corners in scanned samples. By varying the size or number of hexagonal apertures, X-ray transmission through the target can be altered (according to the Beer— Lambert law), enabling a bespoke target that reflects the density and porosity associated with a sample object which is to be imaged by the system. The variant shown in the attached technical drawing is approximately 40% hollow by area, in comparison with 2% for the target of FIG. 1.

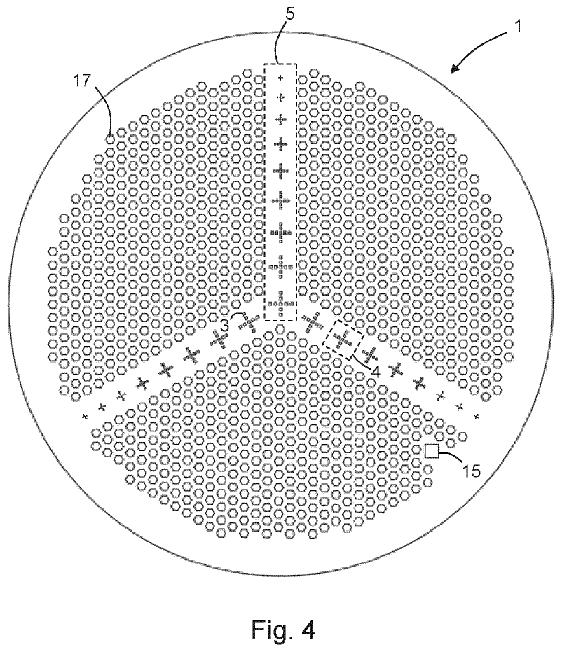

FIG. 4 shows a further variant of the target. In this variant the disc 1 is again perforated with a regular pattern of hexagonal apertures 17 so that the volume of material along X-ray photon paths is similar to that of a hollow object. However, unlike the targets of FIGS. 1 and 3, in this further variant, the columns 3 are arranged in sub-groups 4 which are positioned along just three first group lines 5 which extend radially from the centre of the disc 1 in a tristar-shape such that the three first group lines are angularly spaced 120° apart. With this arrangement of columns 3 the target has a relatively low number of columns and thus can better emulate the X-ray attenuation properties of a solid object to be imaged having little or no voids or porosity.

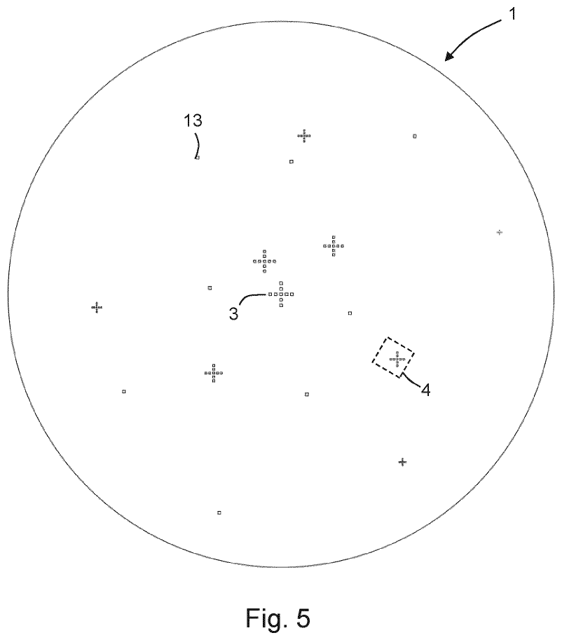

FIG. 5 shows a further variant of the target. In this variant the sub-groups 4 are not arranged along first group lines, but rather are arranged at respective non-periodic angular positions around the centre of the disc 1. The sub-groups 4 form a single set in which the first sub-groups are at respective and different radial distances from the centre, and in which the predetermined transverse diameters d of the first sub-groups vary (in this case increase) with distance from the centre. This arrangement of sub-groups can reduce the number of columns 3 encountered by an X-ray photon along a given ray path, decreasing the interference in the signal response arising from different first sub-groups.

The target also has plural of the further columns 13, and for a similar reason these are arranged at respective non-periodic angular positions around the centre of the disc 1 at respective and different radial distances from the centre.

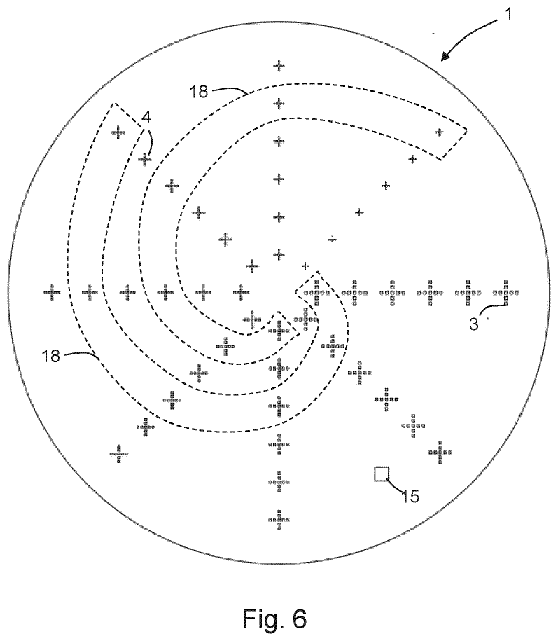

FIG. 6 shows a further variant of the target. In this variant the sub-groups 4 are arranged along respective, equally angularly spaced, radial lines with the transverse diameter d of the columns 3 of each sub-group being the same for all the sub-groups of that radial line. The sub-groups thus form plural spiral sets 18 (two of which is indicated by a respective dashed outline in FIG. 6) in which the first sub-groups are at respective and different radial distances from the centre, and in which the predetermined transverse diameters d of the first sub-groups vary (increase for the two sub-groups indicated) with distance from the centre.

The target also has a square through-hole aperture 15 for orientating image data obtained from the target in virtual space in order to identify feature positions and facilitate the application of relevant profiles.

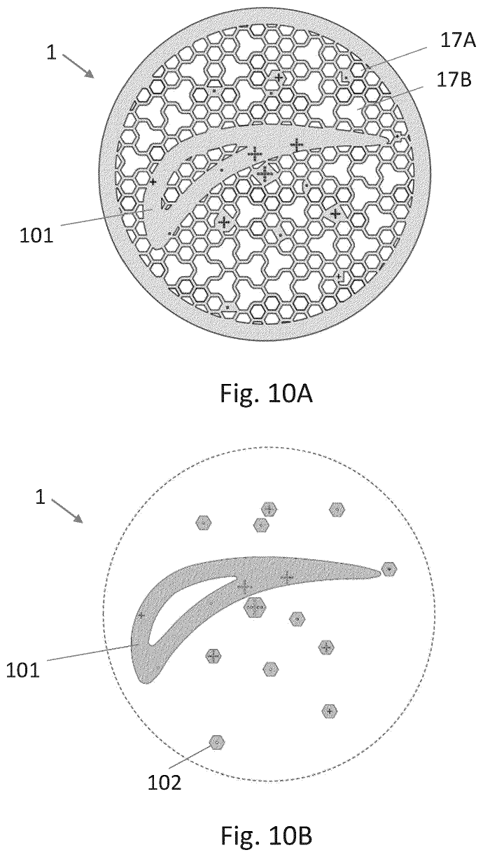

Further variants of the target are shown in FIGS. 10A and 10B. As shown in FIGS. 10A and 10B, in these variants, the target 1 includes radiolucent columns arranged substantially according to the arrangement described above with reference to FIG. 5.

In the variant of FIG. 10A, as in the variants of FIGS. 3 and 4, the disc 1 is perforated with a pattern of apertures. As shown in FIG. 10A, in contrast with the variants of FIGS. 3 and 4, the pattern of apertures in this variant includes apertures of differing sizes and shapes, including hexagonal apertures 17A, and larger apertures 17B that in this example correspond to three of the smaller hexagonal apertures 17A. This can allow a further reduction of a volume of material present, which can facilitate an increased degree of similarity in X-ray attenuation properties between the target and object to be imaged.

In the variant of FIG. 10B, a further reduction in a volume of material present in the imaging plane is achieved by forming the radiolucent columns in radiopaque columns 102 that extend from the disc 1 in the thickness direction.

As shown in FIGS. 10A and 10B, in these variants, the target furthermore includes an imaging structure 101 which shaped to correspond to an object to be imaged, which in this case is a turbine blade. Inclusion of the imaging structure 101 in the target can further increase a degree of similarity in X-ray attenuation properties between the target and object to be imaged.

Target Fabrication

The target and columns can be fabricated by any suitable process, such as 3D printing, additive manufacturing (AM), and lithography.

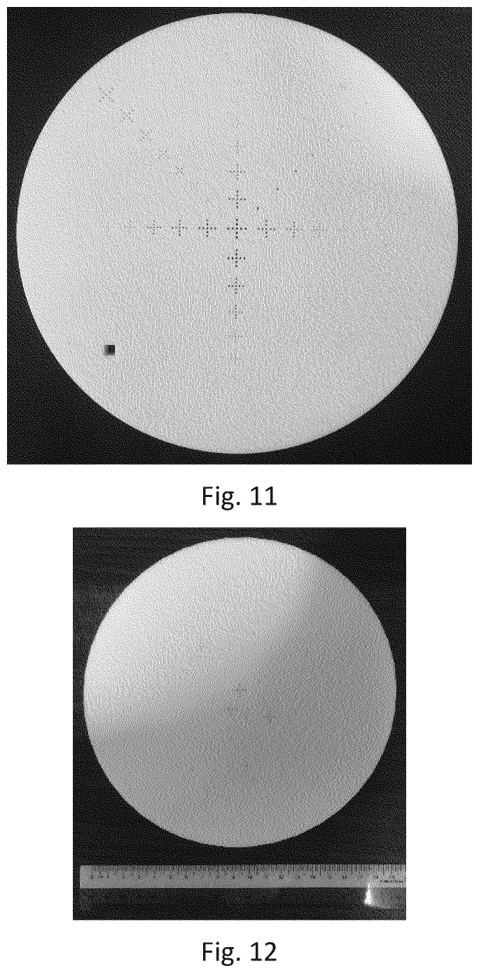

FIG. 11 is a photograph of an example of a plastic target fabricated using 3D printing. In this example, the target is fully dense, has a diameter of 200 mm, and contains radiolucent square cross-section through-holes (columns) arranged substantially according to the arrangement described above with reference to FIG. 1.

FIG. 12 is a photograph of another example of a plastic target fabricated using 3D printing. In this example, the target is fully dense, has a diameter of 200 mm and contains radiolucent square cross-section through-holes (columns) arranged substantially according to the arrangement described above with reference to FIG. 5.

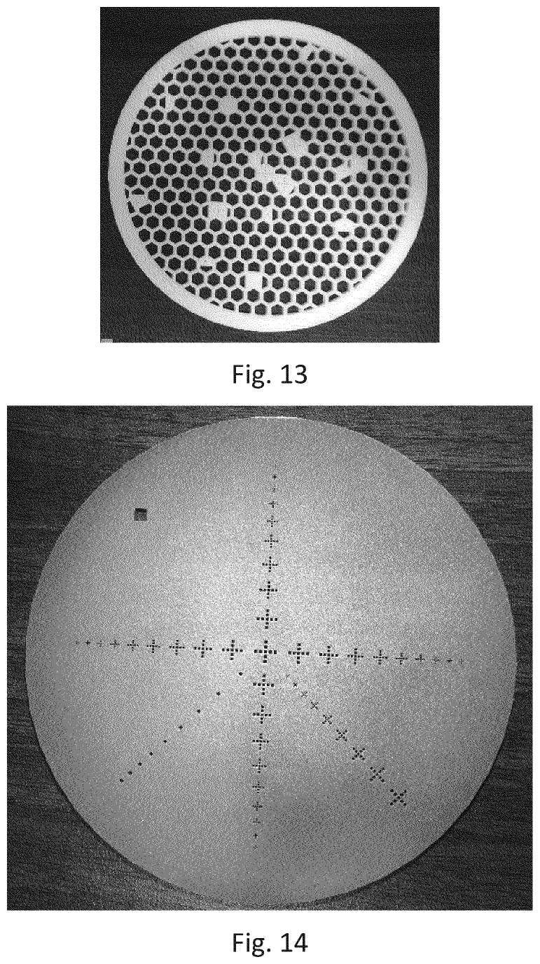

FIG. 13 is a photograph of another example of a plastic target fabricated using 3D printing. In this example, as in the example of FIG. 12, the target has a diameter of 200 mm and contains radiolucent square cross-section through-holes (columns) arranged substantially according to the arrangement described above with reference to FIG. 5. However, in contrast with the example of FIG. 12, the disc of the FIG. 13 example is perforated with a regular pattern of hexagonal apertures, e.g. as described above, such that the overall body density is about 50% of that of the FIG. 12 example.

FIG. 14 is a photograph of an example of a titanium target fabricated using additive manufacturing (AM). In this example, the target is fully dense, has a diameter of 100 mm, and contains radiolucent square cross-section through-holes (columns) arranged substantially according to the arrangement described above with reference to FIG. 1. Targets have been fabricated by lithographically forming columns in a titanium disc. These targets have thicknesses in the range 4 mm to 20 mm, with column transverse diameters, d, in the range 10 microns to 100 microns.

Use of Target

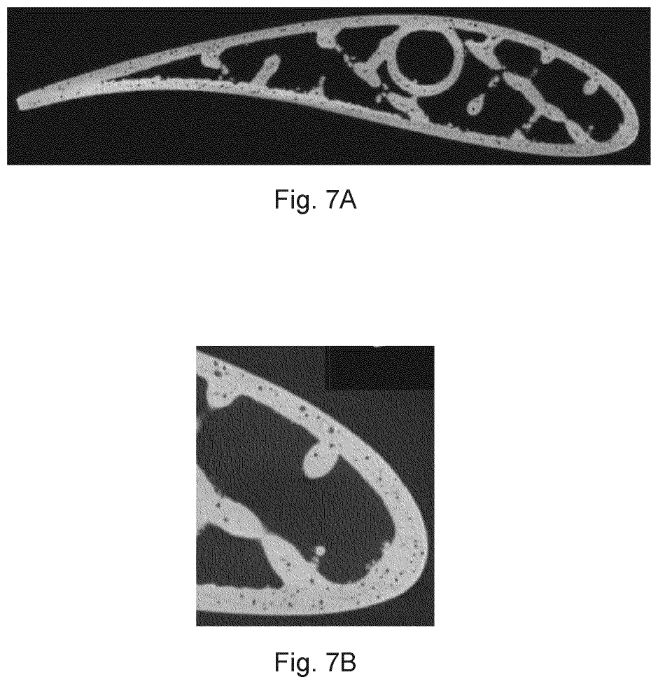

FIG. 7A shows an XCT image of a section through a complex aerofoil produced by additive manufacture (AM), and FIG. 7B shows a close-up view of the leading edge part of the section with porosity identified in red. The AM process enables complex-shaped parts to be formed, but it is necessary to have confidence that such parts comply with quality-control standards. In particular, a part formed with AM can have internal porosity, and standards may stipulate limitations on amounts and type of such porosity. XCT can image internal porosity, and thus can be used to determine compliance with standards, but only if the XCT images themselves can be accurately calibrated and their spatial resolutions determined.

Evaluation of confidence intervals from the calibration target permits the certainty with which features in a real component are recognised as distinct from the accompanying background to be parameterised. Of particular significance are the metrics limit of detection LOD and limit of quantification LOQ, which respectively signify the feature size above which it can be quantitatively assessed whether the feature is present and the feature size above which it is possible to provide repeatable and accurate numerical measurements. LOD and LOQ can be determined by considering the statistical significance of the separation between the values of useful signal and background noise in scanned data of the calibration target. Profiling the signal associated with each of the column sub-groups 4 of the calibration target allows a z value to be assigned to each of the present column size values. Here, z is defined as

Z=(<I_{image}>-<I_{background}>)/σtotal (1)

where <I_{image}> and <I_{background}> respectively represent the mean average intensities of the signal from the columns 3 and background and σₜₒₜₐₗ is defined as the sum of the standard deviations in the column signal and the background signal. These quantities are evaluated by use of line profiles across the sub-groups 4 and background of the 3D calibration target. The z value associated with each column size can be converted to a confidence interval, CI, by use of the error function

CI=erf(z/√2) (2)

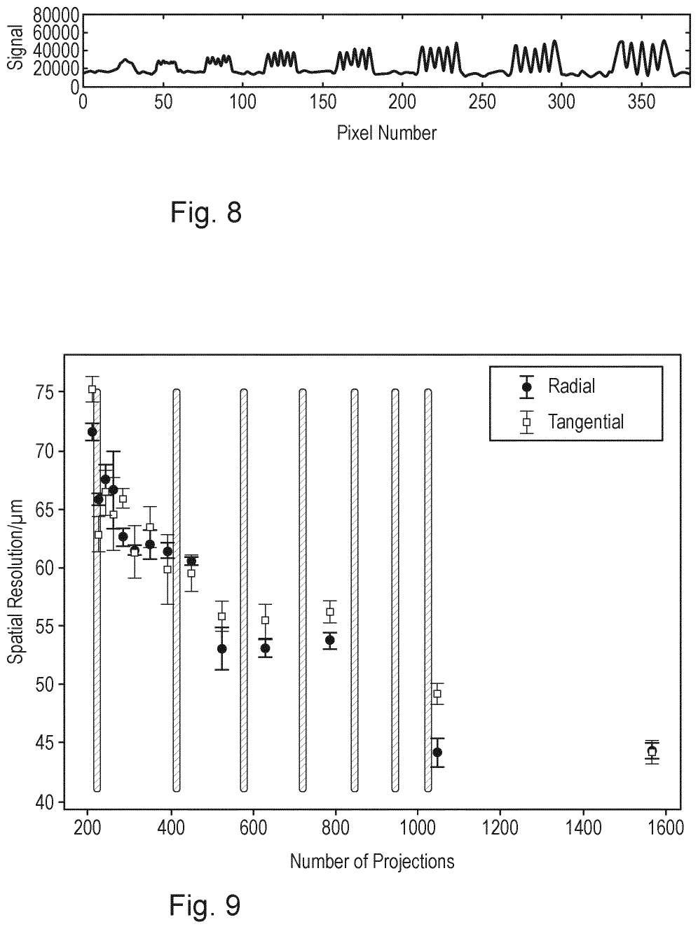

which quantifies the likelihood a column does not belong to the background. A linear least squares fit permits z to be profiled as a function of column size and allows z values to be assigned to size values intermediate and exceeding those found on the calibration target. Equation (2) facilities the conversion of this relationship into CI form, permitting features in the real component to be assigned CI values. Depending on application, threshold CI values can be set to define LOD and LOQ. For instance, the Clinical and Laboratory Standards Institute sets LOD at a z value of 1.645. This corresponds to a CI of 90% and therefore a false positive detection rate of 5%. LOQ is commonly set at a z value of 5, corresponding to a CI of 99.999999% and a false positive detection rate of 5×10⁻⁷%. The target can be used for such calibration and spatial resolution determination. The square through-hole aperture 15 facilitates orientation of the image data obtained from the target in post-processing. In the case of the targets of FIGS. 1, 3 and 4, a primary aim task of the post-processing is then to produce line profiles along each of the first group lines 5. Such a line profile (for eight first sub-groups 4) along the radial direction R of one of the first group lines 5 is shown schematically in FIG. 8. Alternatively, in the case of the variant target of FIG. 5 or FIG. 6, an aim is to produce a line profile through each sub-group 4 of a given set, which can then be combined with the line profiles from the other sub-groups of the set to produce a similar “composite” profile of signal strength against radial distance (which can be converted into an equivalent pixel number).

The values for the transverse diameter d of the first sub-groups 4 decrease from right to left in FIG. 8. At far right, five strong signal peaks centred on pixel number 350 correspond to the five columns 3 of the first column row 9 of the first sub-group with the biggest value of d. Tracking to the left, the signal peaks become progressively weaker and less distinct up to the five signal peaks centred on about pixel number 80. For the final two groups of signal peaks corresponding to the first sub-groups with the smallest values of d, the peaks are merged and no longer distinct. Thus the resolution limit as determined by this line profile is about the value of d corresponding to the first sub-group centred on about pixel number 80. An objective process for identifying the resolution limit can be performed, for example, by making a non-linear least squares Gaussian fit of Michelson contrast as a function of column size to derive a modulation transfer function (MTF) corresponding to the line profile data. However, other approaches can be used to derive the MTF, such as taking the Fourier transform of the derivative of the blurring at edges in an image. The critical size below which the transverse diameter d is considered to be smaller than the resolution limit of the system is dictated by the column size at which the calculated MTF intersects the Michelson contrast value of the background noise. The value of the column size of this intersect is then taken as the spatial resolution.

SNR and CNR are determined through analysis of intensity versus position line profiles obtained from the scanned calibration target.

CNR is the most widely adopted indicator of XCT image quality, owing to its ubiquity within medical images

CNR=|I_{image}-I_{background}|/σ_{N} (3)

where I_{image} is the intensity of the image signal, I_{background} is the intensity of the background signal and σ_{N} is the standard deviation of the noise (assumed here to be the same for the signal and background). This relationship can be exploited to permit calculation of CNR from the described calibration target. I_{image} can be taken to be the mean average value of the signal maxima found in a line profile across a column sub-group, while I_{background} can be taken as the mean average value of a line profile taken across a region of the calibration target devoid of features. σ_{N} is similarly taken as the standard deviation of this background line profile.

SNR, parameterised by the equation

SNR=I_{image}/σ_{N} (4)

is the ratio of the useful signal obtained by an image system to the background noise. SNR can be determined from the calibration target by using the previously outlined method to find I_{image} and σ_{N}. The Rose criterion is a widely used image quality standard and states that for imaged features to be distinguished with near 100% certainty, a CNR/SNR value of at least 5 is required.

Further resolution determinations can be performed for the other first group lines 5, and at different imaging conditions (e.g. numbers of projections used to build up a given XCT image), in order to build up a more complete picture of how resolution is affected by distance from the centre O, angular direction and imaging condition.

Having the value of d reduce with increasing distance from the centre O compliments the Nyquist-Shannon theorem whereby the achievable spatial resolution deteriorates as a function of distance away from the centre O, resulting in an inability to fully resolve features at the largest radial distances. As further imaging projections are removed, this fundamental tenet of image reconstruction results in the loss of resolution moving towards the centre O. The first group lines 5 are arranged to allow a quantification of the number of projections required to resolve a feature of a specific size. Performing XCT with the minimum number of projections needed to resolve features above a given size threshold can save a substantial amount of time in industrial settings.

Line profiles such as that shown in FIG. 8 also provide useful calibration information. For example the full width at half maximum (FWHM) can be determined for all the distinct signal peaks, assuming a Gaussian profile fitted e.g. by a nonlinear least squares method to the peaks. As these peaks derive from columns 3 of known transverse diameter d, it is then possible to plot FWHM against d. The standard deviation in the FWHM values obtained from peaks from columns having an identical transverse diameter d can be assigned as the error associated with that measurement. The presence of a range of d values on the target permits these errors to be plotted as a function of d, and a non-linear least squares fit employed to calculate intermediary values and values outside the measured range. Corrections to the errors obtained in this way can be made for the known actual variations in d values resulting from the manufacturing process. Applied to an image of an object having similarly sized internal features (e.g. porosity) it is then possible to use the plot and measured FWHMs caused by the porosity to calibrate the size of the porosity. Like the spatial resolution, SNR and CNR determinations, further calibrations can be performed for the other first group lines 5, and at different imaging conditions in order to build up a more complete picture of how calibration can be affected by distance from the centre O, angular direction and imaging condition.

Advantageously, both the spatial resolution, SNR and CNR determinations and the calibration can be performed on a target that is of a similar size and has similar attenuation properties as the object to be imaged. In addition, the target allows the effects of different XCT reconstruction codes upon the calibration, spatial resolution, SNR and CNR to be understood. Reconstruction codes use 2D radiographs to predict the position of objects in a virtual 3D volume. Different reconstruction codes address this problem in different ways and utilise different image filters to reduce noise and artefacts. The cross-shape of the first group lines 5 allows the different performances of these reconstruction codes to be quantified. This allows a code to be selected that optimises post-processing efficiency for quality and time in an industrial setting. The target can also be used to study other parameters of the XCT system such as pixel size, X-ray energy, X-ray flux, noise etc.

Similar analyses as those described above on the line profile for the first group lines 5, can be performed on line profiles for the second column rows 11 of the first sub-groups 4 of the first group lines in order to determine spatial resolution, SNR and/or CNR and perform calibration in the azimuthal direction of the disc 1.

An important consideration in XCT is the scan time. This is strongly related to the number of projections used for image reconstruction. The spatial resolution, SNR and CNR evaluation methods described above can be employed to profile the behaviour of these parameters as a function of the number of projections used to reconstruct an XCT dataset acquired from a scan of the target. To illustrate this an experiment was performed in which 1570 projections were gathered in the XCT acquisition phase and subsequent FDK reconstruction was performed upon subsets of these data of varying size, ranging from the full 1570 projections to a minimum of 209 projections. The MTF method of spatial resolution determination was applied to each of these reconstructed datasets, permitting the spatial resolution associated with each number of projections in a given reconstruction to be calculated. The resultant plot of spatial resolution as a function of number of projections is shown in FIG. 9. As expected, spatial resolution improves as the number of projections and therefore the sampling frequency used to reconstruct the image is increased. However, this improvement behaves in a fashion similar to an inverse power relationship, with precipitous gains in image quality observed at low projection numbers and more gradual gains observed at high projection numbers. This suggests the existence of an optimum number of projections to balance image quality against scan time.

In addition to the first group lines 5, the disc 1 also has the 45° second group line 7. Information density from an XCT scan increases from the outside edge of the scan towards the centre of rotation, according to the Nyquist-Shannon theorem, therefore an outside edge feature will always be measured with less information compared to a feature at the centre of the scan. The second group line has its smallest columns 3 close to the centre O and its largest columns 3 at the disc edge. This enables a comparison of the measurement confidence of similarly sized columns 3 at different radial positions to be made (e.g. a column with a small transverse diameter d at the outside edge from one of the first group lines 5 and a similar column towards the centre from the second group line 7). In this way it is possible to determine if a given feature is unresolved because it is too small or if it is too far from the centre O.

Finally, the further square cross-section columns 13 provides an extra line of standard-sized features (all the further columns 13 have a transverse diameter d of 0.10 mm) against which further spatial resolution limits can be determined, as a function of radial distance. The transverse diameter is selected so that the further columns are easily resolvable close to the centre O of the disc 1 and less so close to the edge but the feature size itself is constant (within the accuracy of the manufacturing process used to form the further columns). They can also be used for error estimation.

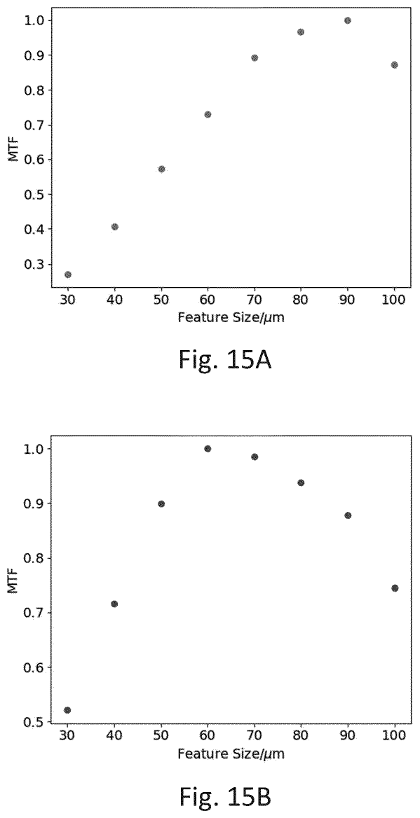

FIG. 15 illustrates an example in which the further square cross-section columns 13 were used to determine correction factors to account for differing levels of noise at different radial distances. FIG. 15A shows an uncorrected plot of MTF versus feature size, and FIG. 15B then shows a plot of MTF versus feature size that has been corrected using correction factors derived from measurements of the further square cross-section columns 13. As can be seen in FIG. 15, the correction in this example results in a shift in the feature size corresponding to maximum MTF.

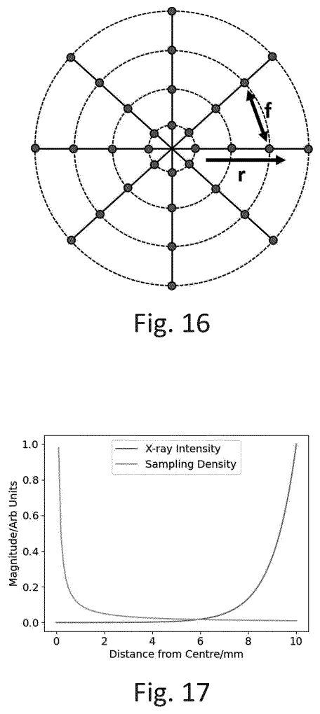

Measurements of e.g. the further square cross-section columns 13 can moreover be used to obtain information regarding the influence of X-ray intensity (signal strength) verses sampling density (radial distance), and the resulting influence on X-ray CT reconstructed spatial resolution. This information can be used to optimise, and ensure consistency of, X-ray CT data collection, e.g. in industrial component Non-Destructive Evaluation applications. For example, FIGS. 16 and 17 schematically illustrate how data density decreases with increasing radial distance from the centre of rotation, whereas X-ray intensity increases with increasing radial distance in X-ray CT, which can result in spatial resolution varying in more complex manner with radial distance. The target can thus be used to determine such variations in spatial resolution. More generally, the target can be used to study scan parameters, and to optimise scan settings. The target can be used to determine a relationship between imaging parameters, such as pixel size, contrast, image noise and reconstruction parameters.

The ability of the target to mimic component designs and radiographic density is particularly advantageous in industrial applications, and can allow tailoring of X-ray CT image quality and measurement confidence to match a component being imaged. The target can accordingly be used both as an Image Quality Indicator (IQI) and Representative Image Indicator (RQI), e.g. in industrial applications.

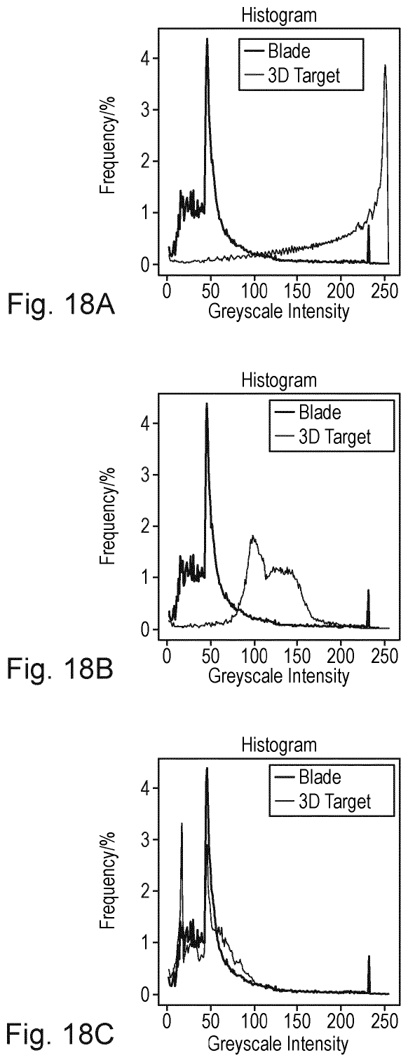

For example, FIG. 18 illustrates a comparison of a histogram of X-ray path lengths for a turbine blade being imaged to histograms of X-ray path lengths for different targets. FIG. 18A shows a comparison between the turbine blade and a fully dense target. FIG. 18B shows a comparison between the turbine blade and the target of FIG. 10A. It will be appreciated by comparing FIGS. 18A and 18B that the X-ray attenuation properties of the target of FIG. 10A are more similar to the turbine blade being imaged than the solid target. FIG. 18C shows a comparison between the turbine blade and the target of FIG. 10B. It will be appreciated by comparing FIGS. 18B and 18C that the X-ray attenuation properties of the target of FIG. 10B are even more similar to the turbine blade.

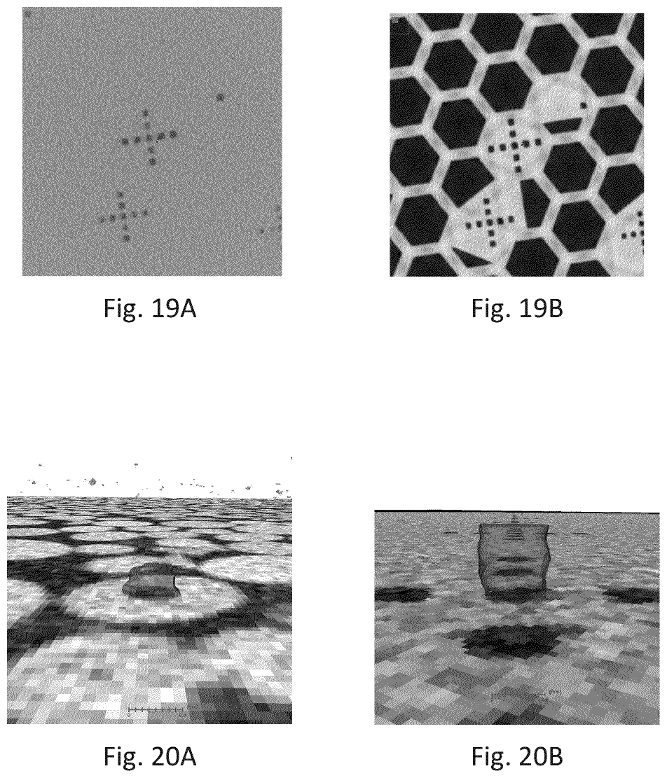

FIG. 19 illustrates a comparison of X-ray CT image quality using the targets shown in FIGS. 12 and 13. FIG. 19A shows an X-ray CT reconstruction of 1000, 900 and 800 micron columns of the fully dense target shown in FIG. 12, and FIG. 19B shows an X-ray CT reconstruction of the corresponding columns of the 50% target shown in FIG. 13. Both targets were imaged at the same time, at 225 keV with 100 micron voxel size. It will be appreciated by comparing FIGS. 19A and 19B that improved image quality and spatial resolution is achieved for the 50% target, due to improved signal strength. This demonstrates that the target can be tailored to achieve an image quality matching that which would be expected for a component being imaged.

FIG. 20 demonstrates that the target can be used to validate measurement confidence for a component being imaged. A 200 mm diameter container of internally porous silica beads was imaged, and FIG. 20A shows a resulting reconstructed orthoslice through one of the beads. The target shown in FIG. 12 was also imaged, and FIG. 20B shows a resulting reconstructed orthoslice through a column of the target that has a similar size to the pore of the bead shown in FIG. 20A. The radiographic transmission properties were similar for both the target and the packed beads. The results allowed a measurement confidence associated with imaging the pore to be validated. The results moreover indicated that although the voxel size was 100 microns, features below 400 microns could not be confidently identified, due to poor signal strength and noise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

1. A target for calibrating and determining the spatial resolution, SNR and/or CNR associated with an X-ray computed tomography system having a rotation axis about which a beam of X-rays is rotated relative to the target to produce signals that are processable to generate an image on an imaging plane perpendicular to the rotation axis,
the target comprising: a radiopaque body (1) and the target being configured to be locatable in the system with a center (O) of the body on the rotation axis, and a thickness direction of the body parallel to the rotation axis such that the body extends radially from its center in the imaging plane;
the body (1) contains a plurality of non-radiopaque columns (3) extending longitudinally in the thickness direction of the body, the columns being arranged in first sub-groups (4) of identically-shaped columns with the columns of each first sub-group sharing a respective predetermined transverse diameter (d), the first sub-groups being spaced from each other, and the columns of each first sub-group also being spaced from each other; and
the first sub-groups are members of one or more first sets, the first sub-groups (4) of each first set being arranged such that within that first set the first sub-groups are at respective and different radial distances from the center (O), and within that first set the predetermined transverse diameters of the first sub-groups vary with radial distance from the center;
such that an image of the target generated by the system is useable to calibrate and determine the spatial resolution, SNR and/or CNR associated with the system on the basis of the predetermined column transverse diameters (d).

2. The target according to claim 1, wherein the columns (3) of each first sub-group (4) are spaced from their nearest neighbour or neighbours by a distance (s) which is in a range from the respective predetermined transverse diameter (d) to three times the respective predetermined transverse diameter, and which is preferably equal to the respective predetermined transverse diameter.

3. The target according to claim 1, wherein the columns (3) are holes extending in the thickness direction, and preferably are through-holes extending across the entire thickness of the body (1).

4. The target according to claim 1, wherein the columns (3) of each first sub-group (4) share a respective predetermined length in the thickness direction, the respective predetermined length being at least three times the respective predetermined transverse diameter (d).

5. The target according to claim 1, wherein the body (1) is planar, and preferably is disc-shaped.

6. The target according to claim 1, wherein the first sub-groups of each first set are arranged along a respective, radially extending, first group line (5).

7. The target according to claim 6 having three first group lines (5) which are formed in a tristar-shape such that the three first group lines are angularly spaced 120° apart, or having four first group lines (5) which are formed in a cross-shape such that the four first group lines are angularly spaced 90° apart.

8. The target according to claim 1, wherein the first sub-groups (4) are arranged within each first set such that the predetermined transverse diameters of the first sub-groups decrease with increasing distance from the centre (O).

9. The target according to claim 8, wherein the body (1) further contains a plurality of further non-radiopaque columns extending longitudinally in the thickness direction, the further columns being arranged in second sub-groups of identically-shaped columns with the columns of each second sub-group sharing a respective predetermined transverse diameter, the second sub-groups being spaced from each other, and the columns of each second sub-group also being spaced from each other;
wherein the second sub-groups are members of one or more second sets, the second sub-groups of each second set being arranged such that within that second set the second sub-groups are at respective and different radial distances from the centre (O), and within that second set the predetermined transverse diameters of the second sub-groups increase with increasing distance from the centre.

10. The target according to claim 9, wherein the second sub-groups of each second set are arranged along a respective, radially extending, second group line (7).

11. The target according to claim 1, wherein the columns of each sub-group (4) are arranged in first (9) and second (11) column rows, the first column row extending along a radial direction (R) of the body (1), and the second column row extending perpendicularly thereto, and preferably wherein the first (9) and second (11) column row of each sub-group (4) form a cross-shape.

12. The target according to claim 1, wherein each column (3) is square prismatic, and thereby intersects as a square cross-section on the imaging plane, the predetermined transverse diameter (d) of each column being equated to the length of the sides of its square cross-section, and preferably wherein two opposing sides of the square cross-section are perpendicular to a radial direction (R) of the body.

13. An assembly comprising:
an X-ray computed tomography system; and
a target, the X-ray computed tomography system having a rotation axis about which a beam of X-rays is rotated relative to the target to produce signals that are processable to generate an image on an imaging plane perpendicular to the rotation axis, and the target calibrating and determining the spatial resolution, SNR and/or CNR associated with the system, wherein the target comprises a radiopaque body and is configured to be locatable in the system with a center of the body on the rotation axis, and a thickness direction of the body parallel to the rotation axis such that the body extends radially from its center in the imaging plane;
the body contains a plurality of non-radiopaque columns extending longitudinally in the thickness direction of the body, the columns being arranged in first sub-groups of identically-shaped columns with the columns of each first sub-group sharing a respective predetermined transverse diameter, the first sub-groups being spaced from each other, and the columns of each first sub-group also being spaced from each other; and
the first sub-groups are members of one or more first sets, the first sub-groups of each first set being arranged such that within that first set the first sub-groups are at respective and different radial distances from the center, and within that first set the predetermined transverse diameters of the first sub-groups vary with radial distance from the center;
such that an image of the target generated by the system is configured to calibrate and determine the spatial resolution, SNR and/or CNR associated with the system on the basis of the predetermined column transverse diameters.

14. The target as claimed in claim 1 wherein the target is used in calibrating and determining respective spatial resolution, SNR and/or CNR associated with one or more X-ray computed tomography systems, each X-ray computed tomography system having a rotation axis about which a beam of X-rays is rotated relative to the target to produce signals that are processable to generate an image on an imaging plane perpendicular to the rotation axis.

15. A method of imaging an object using an X-ray computed tomography system having a rotation axis about which a beam of X-rays is rotated relative to a target to produce signals that are processable to generate an image on an imaging plane perpendicular to the rotation axis, the method including performing the steps of:
a) providing the target and an object to be imaged, the target being formed of a material which has substantially the same attenuation properties to passage of X-rays therethrough as the material of the object;
b) locating the target in the system with the center of its body on the rotation axis, and the thickness direction of the body parallel to the rotation axis such that the body extends radially from its center in the imaging plane, and then generating an image of the target using the system to calibrate and determine the spatial resolution, SNR and/or CNR associated with the system; and
c) locating the object in the system, and then generating an image of the object using the system;
wherein steps (b) and (c) can be performed in any order, and the calibration and spatial resolution, SNR and/or CNR determination resulting from step (b) is applied to the image generated at step (c), and
wherein the target comprises a radiopaque body and is configured to be locatable in the system with a center of the body on the rotation axis, and a thickness direction of the body parallel to the rotation axis such that the body extends radially from its center in the imaging plane;
the body contains a plurality of non-radiopaque columns extending longitudinally in the thickness direction of the body, the columns being arranged in first sub-groups of identically-shaped columns with the columns of each first sub-group sharing a respective predetermined transverse diameter, the first sub-groups being spaced from each other, and the columns of each first sub-group also being spaced from each other; and
the first sub-groups are members of one or more first sets, the first sub-groups of each first set being arranged such that within that first set the first sub-groups are at respective and different radial distances from the center, and within that first set the predetermined transverse diameters of the first sub-groups vary with radial distance from the center;
such that the image of the target generated by the system is useable to calibrate and determine the spatial resolution, SNR and/or CNR associated with the system on the basis of the predetermined column transverse diameters.
